(19) United States
(12) Patent Application Publication
(10) Pub. No.: US 2026/0048069 A1
(43) Pub. Date: Feb. 19, 2026

(54) METHODS OF TREATMENT

(71) Applicant: Arena Pharmaceuticals, Inc., New York, NY (US)

(72) Inventors: Chadwick J. Orevillo, Lebanon, NJ (US); Randall Kaye, New York City, NY (US); Nuggehally Srinivas, Exton, PA (US)

(21) Appl. No.: 19/280,215

(22) Filed: Jul. 25, 2025

Related U.S. Application Data
(60) Provisional application No. 63/675,964, filed on Jul. 26, 2024.

(51) Int. Cl. A61K 31/5517; A61P 25/10; A61P 25/12

(52) U.S. Cl. CPC A61K 31/5517; A61P 25/10; A61P 25/12

(57) ABSTRACT

Provided herein are treatment methods including administering a 5-hydroxytryptamine (HT)_{2C} receptor agonist to a patient in need thereof. An exemplary method includes treating or preventing a 5-hydroxytryptamine (HT)_{2C} receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof.

The developmental and epileptic encephalopathies (DEE) are a heterogeneous group of rare neurodevelopmental disorders. They are characterized by early-onset seizures that are often intractable and are associated with electroencephalographic abnormalities, and developmental delay or regression that can worsen over time. These disorders are generally diagnosed in childhood and adolescence; they vary in their etiologies, seizure types, electroencephalographic patterns, cognitive deficits, and prognosis. The International League Against Epilepsy recently expanded this definition to include disorders that may result in developmental delay before epilepsy onset and used the term of DEE to encompass this broader population.

5-HT2 receptor agonists have been shown to be efficacious treatments for a variety of motor seizures and seizure disorders. Specifically, low dose fenfluramine (Fintepla®), a mixed 5-HT_{2C}, 5-HT_{2B}, and 5-HT_{2A} receptor agonist, has recently been approved for the treatment of Dravet syndrome and Lennox-Gastaut Syndrome. However, Fintepla® received a Boxed Warning requiring cardiac monitoring because of the association between serotonergic drugs with 5-HT_{2B} receptor agonist activity and valvular heart disease.

The 5-HT_{2C} receptor agonist (R)—N-(2,2-difluoroethyl)-7-methyl-1,2,3,4,6,7-hexahydro-[1,4]diazepino[6,7,1-hi]indole-8-carboxamide (see U.S. Pat. No. 10,392,390) is in clinical trials for the treatment of DEE and related seizure disorders. US Pat. App. Pub. 2023/0293546 sets out TID dosing regimens (including up-titration and down-titration) at dosage amounts of 3, 6, 9, 12, 18, and 24 mg and reports PK data including a half-life at steady state ranging from 4.81 to 6.50 hours.

There is a significant unmet need for safe and effective treatments for DEE and other seizure disorders. The compound and dosing methods described herein help satisfy this need and provide related advantages as well.

### SUMMARY

Provided is a method of treating or preventing a 5-hydroxytryptamine (HT)2C receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

Also provided is a method of treating or preventing epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

Also provided is a method of reducing severity of an epileptic seizure in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

Also provided is a method of reducing the frequency of epileptic seizures in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

Also provided is a method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

Also provided is a method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

Also provided is a method of treating or preventing a refractory epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

In some embodiments, Compound 1, or a pharmaceutically acceptable salt thereof, is administered at a dosage equivalent to from about 0.05 to about 0.3 mg/kg Compound 1 free base three times daily (TID).

These and other aspects of the invention disclosed herein will be set forth in greater detail as the patent disclosure proceeds.

### DRAWINGS

FIG. 1 shows the two cohorts in Study 102 (6 mg TID (Upper figure) and 12 mg TID (lower figure)) (Median: Solid line together with its 5th percentile and the 95th percentile (dotted lines). The popPK model predictions are shown in grey with the 95% confidence interval. The y-axis shows Compound 1 (mg/mL) in plasma and the x-axis shows time after first dose (hours).

FIG. 2 shows the change from baseline in frequency of observed countable motor seizures (%) (y-axis) vs the Cavg in plasma (ng/ml) (x-axis) as divided into 4 quartiles (Q) (Q1 (5.92-22.4) Q2 (22.4-31.6) Q3 (31.6-46.2) Q4 (46.2-79.5). N corresponds to the number of patients within a quartile (placebo=9; Q1=9; Q2=9; Q3=8; Q4=9).

FIG. 3 shows the change from baseline in frequency of observed countable motor seizures (%) (y-axis) vs the average in plasma at steady stage of Compound 1 (ng/ml) (x-axis). In grey is shown the 95% confidence interval.

FIG. 4 shows the frequency discontinuations (%) (y-axis) vs the maximum plasma concentrations of Compound 1 (ng/ml) (x-axis) given in quartiles (Q) (Q1 2.54-11.3; Q2 11.3-16.2; Q3 16.2-24.2; Q4 24.2-31.9). Placebo and Q1 had 0 discontinuations, Q2 and Q3 had 2 discontinuations, and Q4 had 7 discontinuations.

FIG. 5 shows the maximum plasma concentration for the first dose of Compound 1 (ng/ml) (y-axis) and the number of patients that completed Study 102 (x-axis) and the number that discontinued.

FIG. 6 shows the fraction of subjects exceeding target exposures within pre-specified body weight groups. Dark grey dots: 50% of patients with Cavg above IC70. Light grey dots: 65% of patients with Cavg above IC50. The figure demonstrates that 12 mg TID appeared to be effective in most adults/subjects above 40 kg, whereas in children/subjects below 40 kg, 12 mg TID appeared to be too high.

FIG. 7 shows a comparison of simulated Cavg in plasma for different body weights and treatment doses. 0.24 mg/kg TID was used if the body weight is below 40 kg, and 12 mg TID was used if the body weight is above 40 kg. Average Compound 1 plasma concentration (ng/ml) is shown on the y-axis and the body weight (kg) is shown on the x-axis. The IC50 is shown on the figure (17.9 ng/mL) as a dotted line. For most subjects below 40 kg the average plasma concentration of Compound 1 was above IC70 of 20.4 ng/ml and IC50. The same is true for patients above 40 kg but below ˜ 90 kg.

FIG. 8 shows a comparison of simulated dose of 0.11 mg/kg TID≤40 kg or 6 mg TID>40 kg for different body weights for Cmax in plasma of the first dose. Maximum Compound 1 plasma concentration (ng/ml) is shown on the y-axis and the body weight (kg) is shown on the x-axis. The limit of 24.2 ng/mL where the number of adverse events increases is shown as a dotted line. The figure shows that not all patients reached a plasma level that corresponds to the IC50 or IC70 values, however all simulations had plasma levels below 24.2 ng/ml, which is the dosage where drop out/side effects become pronounced.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)—N-(2,2-difluoroethyl)-7-methyl-1,2,3,4,6,7-hexahydro-[1,4]diazepino[6,7,1-hi]indole-8-carboxamide.

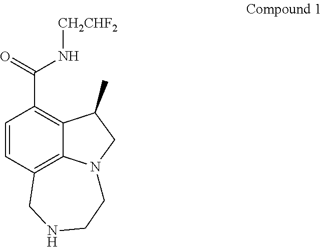

Compound 1, or a pharmaceutically acceptable salt thereof, is a potent and selective 5-hydroxytryptamine (HT)2C receptor agonist and exhibits increased selectivity for the ligand binding site of 5-HT2C receptors versus those of 5-HT2A and 5-HT2B. Compound 1 can also be referred to as “bexicaserin” or “LP352. Compound 1 displays a binding affinity of 44 nM at the human 5-HT2C receptor and shows no activity for 5-HT2A and 5-HT2B, in contrast, for example, to previously developed agonists such as Fintepla® (low dose fenfluramine).

Methods of use of Compound 1, or a pharmaceutically acceptable salt thereof, are disclosed in U.S. Pat. No. 10,392,390, which is incorporated herein by reference in its entirety for all purposes.

CONVULSIVE/MOTOR SEIZURES: As used here, a “convulsive/motor seizure” refers to a tonic-clonic, tonic, tonic-atonic leading to drop, focal motor, epileptic spasms, myoclonic-atonic leading to drop and seizures. Non-convulsive seizures include myoclonic, absence, atypical absence, or atonic seizures and focal seizures without an observable motor component.

CONVULSIVE/MOTOR SEIZURE-FREE DAY: As used herein, a “convulsive/motor seizure-free day” refers to a day for which diary data are available and no convulsive/motor seizures were reported.

DROP SEIZURE: As used herein, the term “drop seizure” refers to a seizure involving the entire body, trunk or head that leads to a fall, injury, slumping in a chair, or head hitting the surface, or could have led to a fall or injury, depending on the position of the subject at the time of the attack or spell.

AGONIST: As used herein, the term “agonist” refers to a moiety that interacts with and activates a receptor, such as the 5-HT_{2C} serotonin receptor, and initiates a physiological or pharmacological response characteristic of that receptor.

ADMINISTERING: As used herein, “administering” means to provide a compound or other therapy, remedy, or treatment such that a patient internalizes a compound.

ORAL or ORALLY: As used herein, “oral” or “orally” refers to administration of a compound or composition to a patient by a route or mode along the alimentary canal. Examples of enteral routes of administration include, without, limitation, oral, as in swallowing solid (e.g., tablet) or liquid (e.g., syrup) forms; sub-lingual (absorption under the tongue); nasojejunal or gastrostomy tubes (into stomach); intraduodenal administration; as well as rectal administration (e.g., suppositories for release and absorption of a compound or composition by in the lower intestinal tract of the alimentary canal).

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to a patient. In this case the health care practitioner may or may not provide a prescription for the compound, dosage regimen, or treatment. Further, the health care practitioner may or may not provide the recommended compound or treatment. For example, the health care practitioner can advise the patient where to obtain the compound without providing the compound. In some embodiments, a health care practitioner can provide a prescription for the compound, dosage regimen, or treatment to the patient. For example, a health care practitioner can give a written or oral prescription to a patient. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the patient. As used herein, giving a sample of a compound constitutes an implicit prescription for the compound. Different health care systems around the world use different methods for prescribing and/or administering compounds or treatments and these methods are encompassed by the disclosure.

A prescription can include, for example, a patient's name and/or identifying information such as date of birth. In addition, for example, a prescription can include: the medication name, medication strength, dosage, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent a patient from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to a patient who may ultimately manifest at least one symptom of a disease or condition but who has not yet done so. Such patients can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to a patient who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

Clinical Global Impression of Severity (CGI-S). According to one embodiment the severity is assessed by CGI-S. The CGI-S according to one embodiment will be rated on a scale from 1 to 7, and evaluated on a an assessment by the doctor “Considering your total clinical experience with this particular population, how ill is the participant at this time?” which is rated on the following 7-point scale: 1=normal, not at all ill; 2=borderline ill; 3=mildly ill; 4=moderately ill; 5=markedly ill; 6=severely ill; 7=among the most extremely ill patients.

According to another embodiment the efficacy may be assessed by Clinical Global Impression of Improvement (CGI-I) and Caregiver Global Impression of Improvement (CaCGI-I). The CGI-I and CaGI-I are assessments of the overall level of improvement. The CGI-I scale will be completed by the doctor, and the CaGI-I scale will be completed by the caregiver. The CGI-I and CaGI-I 7-point scale is as follows: “Very Much Improved” (1); “Much Improved” (2); “Minimally Improved” (3); “No Change” (4); “Minimally Worse” (5); “Much Worse” (6); “Very Much Worse” (7).

TOLERATE: As used herein, a patient is said to “tolerate” a dosage of a compound if administration of that dosage to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate headache, whereas a second patient may find headache tolerable but is not able to tolerate vomiting, whereas for a third patient, either headache alone or vomiting alone is tolerable, but the patient is not able to tolerate the combination of headache and vomiting, even if the severity of each is less than when experienced alone.

INTOLERANCE: As used herein, “intolerance” means significant toxicities and/or tolerability issues that led to a reduction in dosage or discontinuation of the medication. “Intolerance” can be replaced herein with the term “unable to tolerate.”

ADVERSE EVENT: As used herein, an “adverse event” is an untoward medical occurrence that is associated with treatment with Compound, 1, or a pharmaceutically acceptable salt thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc.) that a patient requires or will benefit from treatment. This judgment is made based on a variety of factors that are in the realm of a caregiver's expertise, but that includes the knowledge that the patient is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

PATIENT: As used herein, “patient” means any human. In some embodiments, a human patient is referred to a “subject,” “participant,” or “patient.”

DOSE: As used herein, “dose” means a quantity of Compound 1, or a pharmaceutically acceptable salt thereof, given to the patient for treating or preventing the disease or disorder at one specific time. As used herein, “dosage” refers to the amount of Compound 1, or a pharmaceutically acceptable salt thereof, in one or more doses.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dosage.

PHARMACEUTICAL COMPOSITION: As used herein, “pharmaceutical composition” means a composition comprising at least one active ingredient, such as Compound 1, including but not limited to, salts of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It will be apparent to those skilled in the art that the dosage forms described herein may comprise, as the active component, either Compound 1, or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, patiently or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1, or a pharmaceutically acceptable salt thereof can be separated into two methods; one method reciting prescribing Compound 1, or a pharmaceutically acceptable salt thereof and the other method reciting administering Compound 1, or a pharmaceutically acceptable salt thereof. In addition, for example, a method that recites prescribing Compound 1, or a pharmaceutically acceptable salt thereof and a separate method of the invention reciting administering Compound 1, or a pharmaceutically acceptable salt thereof can be combined into a single method reciting prescribing and/or administering Compound 1, or a pharmaceutically acceptable salt thereof.

### METHODS

Provided is a method of treating or preventing a 5-hydroxytryptamine (HT)2C receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

Also provided is a method of treating or preventing epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

In some embodiments, the epilepsy is a focal epilepsy, a generalized epilepsy, or a combined generalized and focal epilepsy. In some embodiments, the epilepsy has one or more of a structural etiology, a genetic etiology, an infectious etiology, a metabolic etiology, and an immune etiology.

Also provided is a method of reducing severity of an epileptic seizure in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

Also provided is a method of reducing the frequency of epileptic seizures in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

In some embodiments, the epileptic seizure is a focal seizure or a generalized seizure. In some embodiments, the epileptic seizure has one or more of a structural etiology, a genetic etiology, an infectious etiology, a metabolic etiology, and an immune etiology.

Also provided is a method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

In some embodiments, the seizure disorder is a focal seizure disorder, a generalized seizure disorder, or combined generalized and focal seizure disorder. In some embodiments, the seizure disorder has one or more of a structural etiology, a genetic etiology, an infectious etiology, a metabolic etiology, and an immune etiology.

In some embodiments, the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, or EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

Also provided is a method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

In some embodiments, the DEE is chosen from epilepsy syndromes that involve a developmental impairment related to the underlying etiology (e.g., genetic mutation), frequent epileptiform activity, or both. In some embodiments, the DEE is epileptic. Epileptic encephalopathy (EE) can involve frequent epileptiform activity that contributes to an extent of cognitive and behavioral impartments that is more severe than what might be expected based on underlying etiology alone. In some embodiments, the DEE is developmental. Developmental encephalopathy (DE) can involve developmental consequences (e.g., developmental slowing or regression) that arise directly from underlying etiology, and may not involve frequent epileptic activity. In some embodiments, the DEE is both epileptic and developmental, in which developmental and epileptic factors both contribute to encephalopathy.

In some embodiments, the DEE is an early infantile DEE (EIDEE, or Ohtahara syndrome). In some embodiments, the EIDEE is an encephalopathy related to mutations in one or more of ARHGEF9, ARX, BRAT1, CASK, CDKL5, CYFIP2, DMXL2, GNAO1, KCNQ2, KCNT1, NECAP1, NSF, PCDH19, PIGA, PLCB1, PNKP, SCN2A, SCN8A, SIK1, SLC13A5, SLC25A22, SPTAN1, ST3GAL3, STXBP1, TBC1D24, WWOX, GLDC, AMT, GCSH, ALDH7A1, and PNPO. In some embodiments, the EIDEE is an encephalopathy associated with nonketotic hyperglycinemia (NKH; e.g., related to mutations in one or more of GLDC, AMT, GCSH), cortical dysplasia, and mitochondrial disorders.

In some embodiments, the EIDEE is a KCNQ2 encephalopathy, an SCN2A encephalopathy, an SCN8A encephalopathy, or a pyridoxine-dependent epilepsy (PDE).

In some embodiments, the DEE is an infantile epileptic spasms syndrome (IESS), which includes infants who present with epileptic spasms who do not fulfill the criteria for West syndrome. In some embodiments, the IESS is an encephalopathy related to mutations in one or more of AARS, ACADS ALG1, ALG13, ALPL, AMT, ARX, ATP2A2, ATP7A, CACNA1A, CACNA1C, CDKL5, CD99L2, CLCN4, CLCN6, CYFIP1, CYFIP2, DCX, DNM1, EEF1A2, FLNA, FOXG1, GABRE, GCSH, GLDC, GNAO1, GNB1, GPT2, GRIN2A, GRIN2B, HCN1, HEXA, IRF2BPL, KCNB1, KCNJ11, KCNQ2, KCNT1, KIF1A, KMT2D, LIS1 (also referred to as PAFAH1B1), MAGI2, MECP2, MED12, MEF2C, MMACHC MT-ND1, MYO18A, NEDD4L, NF1, NPRL3, NTRK2, PNKP, SCN2A, SCN8A, SCN10A, SETBP1, SETD5, SLC25A22, SLC35A2, SMARCA2, SPTAN1, STXBP1, TAF1, TBL1XR1, TCF4, TCF20, TSC1, TSC2, TUBA1A, UFC1, and WDR45. In some embodiments, the EISS is an encephalopathy related to 17p13.3 microdeletion, Xp22.13 microdeletion, 20q13.33 microdeletion, 9q33.3-34.11 microdeletion, 9p24.3-22.3 microdeletion, 5p12-11 microduplication, 3p25.3 microdeletion, 1p36.33 microdeletion, Xp22.11-21.3 microduplication, or 15q11.2 microduplication.

In some embodiments, the IESS is an encephalopathy associated with chromosome syndromes (e.g., 1p36 deletion syndrome, tetrasomy 12p, dup15q syndrome, trisomy 21 (Down syndrome)), NKH, organic acidemias, hypoxic ischemic encephalopathy (HIE), neurofibromatosis, intracranial infection, brain injury secondary to neonatal hypoglycemia, intracranial hemorrhage (ICH), encephalomalacia, neuroglioma, focal brain lesion, pachygyria-lissencephaly, focal cortical dysplasia, heterotopia, polymicrogyria, schizencephaly, agenesis of the corpus callosum, intracranial hemangioma, Menkes disease, neurodegeneration with brain iron accumulation, methylmalonic acidemia (MMA), short-chain acyl-CoA dehydrogenase (SCAD) deficiency, lysosomal storage diseases, mitochondrial disorders, intra-uterine infection, GLUT-1 deficiency syndrome (hypoglycorrhachia), leukoencephalopathy, and hypophosphatasia.

In some embodiments, the IESS is tuberous sclerosis complex (TSC; associated with mutations in TSC1 or TSC2), an ARX encephalopathy, a CDKL5 encephalopathy (CDK5L deficiency disorder), or an STXBP1 encephalopathy.

In some embodiments, the DEE is chosen from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

In some embodiments, the DEE is chosen from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

Also provided is a method of treating or preventing a refractory epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

In some embodiments, the patient has a comorbid condition, such as intellectual disability, autism spectrum disorder, and/or behavioral problems.

In some embodiments, the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

In some embodiments, the administration results in an improvement in the frequency of convulsive/motor seizures and other seizure types. In some embodiments, the administration results in an improvement in one or more of the following:
frequency of observed countable motor seizures;
number of total seizures;
frequency of non-convulsive seizure;
number of episodes of status epilepticus;
frequency of use of rescue medication; and/or
number of countable motor seizure-free days.

In some embodiments, the administration results in an improvement in Quality of Life Inventory-Disability (QI-Disability), Subject/Caregiver and Investigator Clinical Global Impression of Improvement (CGI-I), the Investigator Clinical Global Impression of Severity (CGI-S), CaGI-I, Neurobehavioral Evaluation Tool (NET), and/or the 55-item Quality of Life in Childhood Epilepsy Questionnaire (QOLCE-55). In some embodiments, the administration results in at least a 1-point change from baseline in CGI-I and/or CGI-S.

In some embodiments, prior to administration, the patient had treatment resistant countable motor seizures with an average of ≥4 observed/countable motor seizures per 4-week period while on stable ASM treatment.

In some embodiments, the patient has a DEE but does not have Dravet syndrome or Lennox-Gastaut Syndrome.

In some embodiments wherein the patient has a DEE but does not have Dravet syndrome or Lennox-Gastaut Syndrome, the patient had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

In some embodiments, the patient has Dravet syndrome.

In some embodiments wherein the patient has Dravet syndrome, prior to administration, the patient had:
onset of seizures between 1 and 20 months of age in an otherwise healthy infant; and/or
prolonged generalized tonic-clonic, hemiclonic, myoclonic, tonic, atonic, atypical absence, focal impaired awareness, and/or nonconvulsive status epilepticus.

In some embodiments wherein the patient has Dravet syndrome, prior to administration, the patient had:
an emergence of another seizure type;
prolonged exposure to warm temperatures-induced seizures and/or seizures that were associated with fevers due to illness or vaccines, hot baths, high levels of activity, and sudden temperature changes; and/or
seizures induced by strong natural and/or fluorescent lighting.

In some embodiments wherein the patient has Dravet syndrome, prior to administration, the patient had genetic test results consistent with a diagnosis of Dravet syndrome.

In some embodiments, the patient has Lennox-Gastaut Syndrome.

In some embodiments, the patient has Lennox-Gastaut Syndrome, prior to administration, the patient had:
a history of tonic seizures or tonic/atonic seizures;
more than 1 type of generalized seizure, including but not limited to atypical absence, atonic, myoclonic, focal impaired awareness, generalized tonic-clonic, nonconvulsive status epilepticus, or epileptic spasms;
a history of seizure before 8 years of age;
a history of developmental delay;
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

In some embodiments, Compound 1, or a pharmaceutically acceptable salt thereof, is administered via a titration scheme that comprises the up-titration of Compound 1, or a pharmaceutically acceptable salt thereof, until an optimized dosage is administered.

In some embodiments, the titration scheme comprises administering Compound 1, or a pharmaceutically acceptable salt thereof, at an initial dosage equivalent to about 0.11 mg/kg of Compound 1 TID and, provided that the patient tolerates the initial dosage and that the patient has not had an adequate response, increasing the dosage.

In some embodiments, the patient is less than or equal to 2 years of age and is administered Compound 1, or a pharmaceutically acceptable salt thereof, at an initial dosage equivalent to about 0.11 mg/kg of Compound 1 TID.

In some embodiments, the increased dosage is equivalent to about 0.17 mg/kg of Compound 1 TID.

In some embodiments, if the patient does not tolerate the increased dosage, the optimized dosage is the initial dosage.

In some embodiments, if the patient tolerates the increased dosage and if the patient has had an adequate response, the optimized dosage is the increased dosage.

In some embodiments, the titration scheme comprises further increasing the dosage, provided that the patient tolerates the increased dosage and that the patient has not had an adequate response.

In some embodiments, the further increased dosage is equivalent to about 0.24 mg/kg Compound 1 free base three times daily (TID).

In some embodiments, if the patient tolerates the further increased dosage and if the patient has had an adequate response, the optimized dosage is the further increased dosage (or maintenance dose selected from 0.11 mg/kg, 0.17 mg/kg or 0.24 mg/kg.).

In some embodiments, the method further comprises administering the optimized dosage of Compound 1, or a pharmaceutically acceptable salt thereof, to the patient.

In some embodiments, the dosage of Compound 1, or a pharmaceutically acceptable salt thereof, is down-titrated to address an observed side effect. In some embodiments, the dosage of Compound 1, or a pharmaceutically acceptable salt thereof, is down-titrated to minimize the risk of a withdrawal induced side effect.

In some embodiments, the dosage of Compound 1, or a pharmaceutically acceptable salt thereof, is gradually increased, i.e., up-titrated, to achieve the highest maintenance dose based on tolerability.

In some embodiments, the patient is less than 2 years of age. In some embodiments, the patient is less than 2 years of age and the maintenance dose is equivalent to up to about 0.3 mg/kg TID Compound 1 free base three times daily.

In some embodiments, the patient is an infant (29 days to less than 2 years). In some embodiments, the patient is an infant (29 days to less than 2 years) and is administered Compound 1, or a pharmaceutically acceptable salt thereof, at a dosage equivalent to from about 0.03 to about 0.05 mg/kg Compound 1 free base three times daily (TID) as the initial dose.

In some embodiments, the patient is less than 2 years of age and is administered Compound 1, or a pharmaceutically acceptable salt thereof, at a dosage equivalent to about 0.03 mg/kg Compound 1 free base three times daily (TID). In some embodiments, the patient is less than 2 years of age and is administered Compound 1, or a pharmaceutically acceptable salt thereof, at a dosage equivalent to about 0.05 mg/kg Compound 1 free base three times daily (TID) as the initial dose.

In some embodiments, the patient is an infant (29 days to less than 2 years) and has body weight of approximately 3 to 10 kg. In some embodiments, the patient is an infant boy and has a body weight between about 10.6 kg and about 15.2 kg. In some embodiments, the patient is an infant female and has a body weight between about 10.2 kg and 14.6 kg.

In some embodiments, the patient is a child (2 years to less than 12 years).

In some embodiments, the patient is an adolescent (12 years to 21 years (up to but not including the 22^{nd} birthday).

In some embodiments, the patient is an adult (22 years or older).

In some embodiments, the patient is ≤40 kg and Compound 1, or a pharmaceutically acceptable salt thereof, is administered at a dose equivalent to Compound 1 free base TID according to the following schedule:

| | | Initial | | Up- | | Up- | | Up- | | Up- |
|---|---|---|---|---|---|---|---|---|---|---|
| Age | | dose | | titration | | titration | | titration | | titration |
| stratification* | | (TID) | | (TID) | | (TID) | | (TID) | | (TID) |
| 0-<2 | years | 0.03-0.05 | mg/kg | 0.07-0.9 | mg/kg | 0.11 | mg/kg | 0.17 | mg/kg | 0.24 mg/kg |
| 2-5 | years | 0.11 | mg/kg | 0.17 | mg/kg | 0.24 | mg/kg | 0.30 | mg/kg | — |
| 6-11 | years | 0.11 | mg/kg | 0.17 | mg/kg | 0.24 | mg/kg | 0.30 | mg/kg | — |
| 12 < 18 | years | 0.11 | mg/kg | 0.17 | mg/kg | 0.24 | mg/kg | 0.30 | mg/kg | — |

Bodyweight based dosing for ≤40 kg and fixed dosing if >40 kg bodyweight

In some embodiments the patient is below 2 years and receive an initial dosage of TID 0.03-0.05 mg/kg followed by an up-titration TID dose selected from 0.07-0.9 mg/kg, 0.11 mg/kg, 0.17 mg/kg and 0.24 mg/kg. The up titration is performed as a dose escalation, wherein the dose is increased until an adequate response is achieved or the dose is not tolerated due to side effects. The highest effective dose will be the maintenance dose, that is the TID maintenance dose is selected from 0.11 mg/kg, 0.17 mg/kg and 0.24 mg/kg.

In some embodiments patients above 2 years, but having a body weight below 40 kg (such as 2-5 years, 6-11 years or 12-18 years) will receive an initial dose of 0.11 mg/kg TID followed by an up-titration TID dose selected from 0.17 mg/kg, 0.24 mg/kg, and 0.30 mg/kg. The up titration is performed as a dose escalation, wherein the dose is increased until an adequate response is achieved or the dose is not tolerated due to side effects. The highest effective dose will be the maintenance dose, that is the TID maintenance dose is selected from 0.17 mg/kg, 0.24 mg/kg, and 0.30 mg/kg.

In some embodiments, the patient is also being administered an antiepileptic drug or antiseizure medicine. In some embodiments, the patient is also being administered an antiepileptic drug effective in suppressing interictal epileptiform discharges (e.g., benzodiazepines, valproic acid, and lamotrigine). In some embodiments, the patient is also being administered an immunomodulatory therapy (e.g., corticosteroids, intravenous immunoglobulin [IVIG], plasmapheresis). In some embodiments, the patient is also being administered a ketogenic diet. In some embodiments, the patient is also being administered vagal nerve stimulation (VNS) or deep brain stimulation (DBS).

In some embodiments, Compound 1, or a pharmaceutically acceptable salt thereof, is administered as a raw or pure chemical, for example as a powder in capsule formulation.

In some embodiments, Compound 1, or a pharmaceutically acceptable salt thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

In some embodiments, the Compound 1, or a pharmaceutically acceptable salt thereof, is formulated in a manner suitable for oral administration.

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

According to one embodiment the Compound 1 as a HCl salt is in a liquid formulation comprising maltodextrin, glycerin, sacralose, sodium benzoate, methylparaben and diluted HCl.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For oral administration, the pharmaceutical composition may be in the form of suitable for administration via gastrostomy tube or percutaneous endoscopic gastrostomy tube.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### Further Embodiments of the Invention

1. A method of treating or preventing a 5-hydroxytryptamine (HT)2C receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

2. A method of treating or preventing epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

3. A method of reducing severity of an epileptic seizure in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

4. A method of reducing the frequency of epileptic seizures in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

5. A method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

6. The method of embodiment 5, wherein the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

7. A method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤40 kg.

8 The method of embodiment 7, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

9. The method of embodiment 7, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

10. A method of treating or preventing a refractory epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the patient has a body weight of ≤ 40 kg.

11. The method of any one of embodiments 1 to 10, wherein Compound 1, or a pharmaceutically acceptable salt thereof, is administered via a titration scheme that comprises the up-titration of Compound 1, or a pharmaceutically acceptable salt thereof, until an optimized dosage is administered.

12. The method of embodiment 11, wherein the titration scheme comprises administering Compound 1, or a pharmaceutically acceptable salt thereof, at an initial dosage equivalent to about 0.11 mg/kg of Compound 1 TID and, provided that the patient tolerates the initial dosage and that the patient has not had an adequate response, increasing the dosage.

13. The method of embodiment 12, wherein the patient is less than or equal to 2 years of age.

14. The method of embodiment 12, wherein the increased dosage is equivalent to about 0.17 mg/kg of Compound 1 TID.

15. The method of embodiment 12 or 13, wherein if the patient does not tolerate the increased dosage, the optimized dosage is the initial dosage.

16. The method of embodiment 12 or 13, wherein if the patient tolerates the increased dosage and if the patient has had an adequate response, the optimized dosage is the increased dosage.

17. The method of any one of embodiments 11 to 15, wherein the titration scheme comprises further increasing the dosage, provided that the patient tolerates the increased dosage and that the patient has not had an adequate response.

18. The method of embodiment 16, wherein the further increased dosage is equivalent to about 0.24 mg/kg Compound 1 free base three times daily (TID).

19. The method of embodiment 17, wherein if the patient tolerates the further increased dosage and if the patient has had an adequate response, the optimized dosage is the further increased dosage or maintenance dose selected from 0.11 mg/kg, 0.17 mg/kg or 0.24 mg/kg.

20. The method of any one of embodiments 11, 15, and 18, further comprising administering the optimized dosage of Compound 1, or a pharmaceutically acceptable salt thereof, to the patient.

21. The method of any one of embodiments 11 to 19, wherein the titration scheme further comprises the down-titration of Compound 1, or a pharmaceutically acceptable salt thereof.

22. The method of embodiment 11, wherein the patient is ≤40 kg and Compound 1, or a pharmaceutically acceptable salt thereof, is administered TID at a dosage equivalent to Compound 1 free base according to the following schedule:

| Age | | Initial dose | | Up- | | Up- | | Up- | | Up- |
|---|---|---|---|---|---|---|---|---|---|---|
| stratification* | | (TID) | | titration | | titration | | titration | | titration |
| 0-<2 | years | 0.03-0.05 | mg/kg | 0.07-0.9 | mg/kg | 0.11 | mg/kg | 0.17 | mg/kg | 0.24 mg/kg |
| 2-5 | years | 0.11 | mg/kg | 0.17 | mg/kg | 0.24 | mg/kg | 0.30 | mg/kg | — |
| 6-11 | years | 0.11 | mg/kg | 0.17 | mg/kg | 0.24 | mg/kg | 0.30 | mg/kg | — |
| 12 < 18 | years | 0.11 | mg/kg | 0.17 | mg/kg | 0.24 | mg/kg | 0.30 | mg/kg | — |

23. The method of any one of embodiments 1 to 22, wherein the administration results in an improvement in the frequency of convulsive/motor seizures.

24. The method of embodiment 23, wherein the administration results in an improvement in one or more of:
frequency of observed countable motor seizures;
number of total seizures;
frequency of non-convulsive seizure;
number of episodes of status epilepticus;
frequency of use of rescue medication; and
number of countable motor seizure-free days.

25. The method of any one of embodiments 1 to 24, wherein the administration results in an improvement in the Quality of Life Inventory-Disability (QI-Disability), Subject/Caregiver and Investigator Clinical Global Impression of Improvement (CGI-I), the Investigator Clinical Global Impression of Severity (CGI-S), CaGI-I, Neurobehavioral Evaluation Tool (NET), and/or the 55-item Quality of Life in Childhood Epilepsy Questionnaire (QOLCE-55).

26. The method of embodiment 25, wherein the administration results in at least a 1-point change from baseline in CGI-I and/or CGI-S.

27. The method of any one of embodiments 1 to 26, wherein prior to administration, the patient had treatment resistant countable motor seizures with an average of ≥4 observed/countable motor seizures per 4-week period while on stable ASM treatment.

28. The method of any one of embodiments 1 to 27, wherein the patient has a DEE but does not have Dravet syndrome or Lennox-Gastaut Syndrome.

29. The method of embodiment 28, wherein prior to administration, the patient had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

30. The method of any one of embodiments 1 to 27, wherein the patient has Dravet syndrome.

31. The method of embodiment 30, wherein prior to administration, the patient had:
onset of seizures between 1 and 20 months of age in an otherwise healthy infant; and/or
prolonged generalized tonic-clonic, hemiclonic, myoclonic, tonic, atonic, atypical absence, focal impaired awareness, and/or nonconvulsive status epilepticus.

32. The method of embodiment 30 or 31, wherein prior to administration, the patient had:
an emergence of another seizure type;
prolonged exposure to warm temperatures-induced seizures and/or seizures that were associated with fevers due to illness or vaccines, hot baths, high levels of activity, and/or sudden temperature changes; and/or
seizures induced by strong natural and/or fluorescent lighting.

33. The method of any one of embodiments 30 to 32, wherein prior to administration, the patient had genetic test results consistent with a diagnosis of Dravet syndrome.

34. The method of any one of embodiments 1 to 27, wherein the patient has Lennox-Gastaut Syndrome.

35. The method of embodiment 34, wherein prior to administration, the patient had:
a history of tonic seizures or tonic/atonic seizures;
more than 1 type of generalized seizure, including but not limited to atypical absence, atonic, myoclonic, focal impaired awareness, generalized tonic-clonic, nonconvulsive status epilepticus, or epileptic spasms;
a history of seizure before 8 years of age;
a history of developmental delay;
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

36. The method of any one of embodiments 1 to 35, wherein the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

37. The method of any one of embodiments 1 to 36, wherein the Compound 1, or a pharmaceutically acceptable salt thereof, is an HCl salt of Compound 1.

38. The method of any one of embodiments 1 to 37, wherein the patient is an infant (less than 2 years of age).

39. The method of any one of embodiments 1 to 37, wherein the patient is a child (2 years to less than 12 years).

40. The method of any one of embodiments 1 to 37, wherein the patient is an adolescent (12 years to 21 years (up to but not including the 22^{nd} birthday)).

41. The method of any one of embodiments 1 to 37, wherein the patient is an adult (22 years or older).

### Further Embodiments of the Invention

1. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof for use in treating or preventing a 5-hydroxytryptamine (HT)2C receptor-associated disorder, wherein the use comprises administering to the patient Compound 1, or a pharmaceutically acceptable salt thereof,
at a dosage equivalent to from about 0.03 to about 0.3 mg/kg of Compound 1 free base three times daily (TID), provided that the patient has a body weight of ≤40 kg.

2. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for use in treating or preventing epilepsy in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

3. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for use in reducing severity of an epileptic seizure in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

4. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for use in reducing the frequency of epileptic seizures in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

5. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for use in treating or preventing a seizure disorder in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

6. The use according to embodiment 5, wherein the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

7. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for use in treating or preventing developmental and epileptic encephalopathy (DEE) in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

8. The use according to embodiment 7, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

9. The use according to embodiment 7, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

10. (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for use in treating or preventing a refractory epilepsy in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

11. The use according to any one of embodiments 1 to 10, wherein Compound 1, or a pharmaceutically acceptable salt thereof, is administered via a titration scheme that comprises the up-titration of Compound 1, or a pharmaceutically acceptable salt thereof, until an optimized dosage is administered.

12. The use according to embodiment 11, wherein the titration scheme comprises administering Compound 1, or a pharmaceutically acceptable salt thereof, at an initial dosage equivalent to about 0.11 mg/kg of Compound 1 TID and, provided that the patient tolerates the initial dosage and that the patient has not had an adequate response, increasing the dosage.

13. The use according to 12, wherein the patient is less than or equal to 2 years of age.

14. The use according to embodiment 12, wherein the increased dosage is equivalent to about 0.17 mg/kg of Compound 1 TID.

15. The use according to embodiment 12 or 13, wherein if the patient does not tolerate the increased dosage, the optimized dosage is the initial dosage.

16. The use according to embodiment 12 or 13, wherein if the patient tolerates the increased dosage and if the patient has had an adequate response, the optimized dosage is the increased dosage.

17. The use according to any one of embodiments 11 to 15, wherein the titration scheme comprises further increasing the dosage, provided that the patient tolerates the increased dosage and that the patient has not had an adequate response.

18. The use according to embodiment 16, wherein the further increased dosage is equivalent to about 0.24 mg/kg Compound 1 free base three times daily (TID).

19. The use according to embodiment 17, wherein if the patient tolerates the further increased dosage and if the patient has had an adequate response, the optimized dosage is the further increased dosage or maintenance dose selected from 0.11 mg/kg, 0.17 mg/kg or 0.24 mg/kg.

20. The use according to any one of embodiments 11, 15, and 18, further comprising administering the optimized dosage of Compound 1, or a pharmaceutically acceptable salt thereof, to the patient.

21. The use according to any one of embodiments 11 to 19, wherein the titration scheme further comprises the down-titration of Compound 1, or a pharmaceutically acceptable salt thereof.

22. The use according to embodiment 11, wherein the patient is ≤40 kg and Compound 1, or a pharmaceutically acceptable salt thereof, is administered TID at a dosage equivalent to Compound 1 free base according to the following schedule:

| Age | | Initial dose | | Up- | | Up- | | Up- | | Up- |
|---|---|---|---|---|---|---|---|---|---|---|
| stratification* | | (TID) | | titration | | titration | | titration | | titration |
| 0-<2 | years | 0.03-0.05 | mg/kg | 0.07-0.9 | mg/kg | 0.11 | mg/kg | 0.17 | mg/kg | 0.24 mg/kg |
| 2-5 | years | 0.11 | mg/kg | 0.17 | mg/kg | 0.24 | mg/kg | 0.30 | mg/kg | — |
| 6-11 | years | 0.11 | mg/kg | 0.17 | mg/kg | 0.24 | mg/kg | 0.30 | mg/kg | — |
| 12 < 18 | years | 0.11 | mg/kg | 0.17 | mg/kg | 0.24 | mg/kg | 0.30 | mg/kg | — |

23. The use according to any one of embodiments 1 to 22, wherein the administration results in an improvement in the frequency of convulsive/motor seizures.

24. The use according to embodiment 23, wherein the administration results in an improvement in one or more of:
frequency of observed countable motor seizures;
number of total seizures;
frequency of non-convulsive seizure;
number of episodes of status epilepticus;
frequency of use of rescue medication; and
number of countable motor seizure-free days.

25. The use according to any one of embodiments 1 to 24, wherein the administration results in an improvement in the Quality of Life Inventory-Disability (QI-Disability), Subject/Caregiver and Investigator Clinical Global Impression of Improvement (CGI-I), the Investigator Clinical Global Impression of Severity (CGI-S), CaGI-I, Neurobehavioral Evaluation Tool (NET), and/or the 55-item Quality of Life in Childhood Epilepsy Questionnaire (QOLCE-55).

26. The use according to embodiment 25, wherein the administration results in at least a 1-point change from baseline in CGI-I and/or CGI-S.

27. The use according to any one of embodiments 1 to 26, wherein prior to administration, the patient had treatment resistant countable motor seizures with an average of ≥4 observed/countable motor seizures per 4-week period while on stable ASM treatment.

28. The use according to any one of embodiments 1 to 27, wherein the patient has a DEE but does not have Dravet syndrome or Lennox-Gastaut Syndrome.

29. The use according to embodiment 28, wherein prior to administration, the patient had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

30. The use according to any one of embodiments 1 to 27, wherein the patient has Dravet syndrome.

31. The use according to embodiment 30, wherein prior to administration, the patient had:
onset of seizures between 1 and 20 months of age in an otherwise healthy infant; and/or
prolonged generalized tonic-clonic, hemiclonic, myoclonic, tonic, atonic, atypical absence, focal impaired awareness, and/or nonconvulsive status epilepticus.

32. The use according to embodiment 30 or 31, wherein prior to administration, the patient had:
an emergence of another seizure type;
prolonged exposure to warm temperatures-induced seizures and/or seizures that were associated with fevers due to illness or vaccines, hot baths, high levels of activity, and/or sudden temperature changes; and/or
seizures induced by strong natural and/or fluorescent lighting.

33. The use according to any one of embodiments 30 to 32, wherein prior to administration, the patient had genetic test results consistent with a diagnosis of Dravet syndrome.

34. The use according to any one of embodiments 1 to 27, wherein the patient has Lennox-Gastaut Syndrome.

35. The use according to embodiment 34, wherein prior to administration, the patient had:
a history of tonic seizures or tonic/atonic seizures;
more than 1 type of generalized seizure, including but not limited to atypical absence, atonic, myoclonic, focal impaired awareness, generalized tonic-clonic, nonconvulsive status epilepticus, or epileptic spasms;
a history of seizure before 8 years of age;
a history of developmental delay;
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

36. The use according to any one of embodiments 1 to 35, wherein the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

37. The use according to any one of embodiments 1 to 36, wherein the Compound 1, or a pharmaceutically acceptable salt thereof, is an HCl salt of Compound 1.

38. The use according to any one of embodiments 1 to 37, wherein the patient is an infant (less than 2 years of age).

39. The use according to any one of embodiments 1 to 37, wherein the patient is a child (2 years to less than 12 years).

40. The use according to any one of embodiments 1 to 37, wherein the patient is an adolescent (12 years to 21 years (up to but not including the 22^{nd} birthday)).

41. The use according to any one of embodiments 1 to 37, wherein the patient is an adult (22 years or older).

### Further Embodiments of the Invention

1. Use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof for the manufacturing of a medicament for use in treating or preventing a 5-hydroxytryptamine (HT)2C receptor-associated disorder, wherein the use comprises administering to the patient Compound 1, or a pharmaceutically acceptable salt thereof,
at a dosage equivalent to from about 0.03 to about 0.3 mg/kg of Compound 1 free base three times daily (TID), provided that the patient has a body weight of ≤40 kg.

2. Use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament for use in treating or preventing epilepsy in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

3. Use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament for use in reducing severity of an epileptic seizure in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

4. Use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament for use in reducing the frequency of epileptic seizures in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

5. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament for use in treating or preventing a seizure disorder in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

6. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 5, wherein the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

7. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament for use in treating or preventing developmental and epileptic encephalopathy (DEE) in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

8. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 7, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

9. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 7, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

10. Use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament for use in treating or preventing a refractory epilepsy in a patient, wherein the use comprises administering to the patient Compound 1, or a pharmaceutically acceptable salt thereof,
at a dosage equivalent to from about 0.03 to about 0.3 mg/kg Compound 1 free base three times daily (TID), provided that the patient has a body weight of ≤40 kg.

11. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 10, wherein Compound 1, or a pharmaceutically acceptable salt thereof, is administered via a titration scheme that comprises the up-titration of Compound 1, or a pharmaceutically acceptable salt thereof, until an optimized dosage is administered.

12. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 11, wherein the titration scheme comprises administering Compound 1, or a pharmaceutically acceptable salt thereof, at an initial dosage equivalent to about 0.11 mg/kg of Compound 1 TID and, provided that the patient tolerates the initial dosage and that the patient has not had an adequate response, increasing the dosage.

13. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to 12, wherein the patient is less than or equal to 2 years of age.

14. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 12, wherein the increased dosage is equivalent to about 0.17 mg/kg of Compound 1 TID.

15. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 12 or 13, wherein if the patient does not tolerate the increased dosage, the optimized dosage is the initial dosage.

16. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 12 or 13, wherein if the patient tolerates the increased dosage and if the patient has had an adequate response, the optimized dosage is the increased dosage.

17. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 11 to 15, wherein the titration scheme comprises further increasing the dosage, provided that the patient tolerates the increased dosage and that the patient has not had an adequate response.

18. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 16, wherein the further increased dosage is equivalent to about 0.24 mg/kg Compound 1 free base three times daily (TID).

19. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 17, wherein if the patient tolerates the further increased dosage and if the patient has had an adequate response, the optimized dosage is the further increased dosage or maintenance dose selected from 0.11 mg/kg, 0.17 mg/kg or 0.24 mg/kg.

20. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 11, 15, and 18, further comprising administering the optimized dosage of Compound 1, or a pharmaceutically acceptable salt thereof, to the patient.

21. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 11 to 19, wherein the titration scheme further comprises the down-titration of Compound 1, or a pharmaceutically acceptable salt thereof.

22. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 11, wherein the patient is ≤40 kg and Compound 1, or a pharmaceutically acceptable salt thereof, is administered TID at a dosage equivalent to Compound 1 free base according to the following schedule:

| Age | | Initial dose | | Up- | | Up- | | Up- | | Up- |
|---|---|---|---|---|---|---|---|---|---|---|
| stratification* | | (TID) | | titration | | titration | | titration | | titration |
| 0-<2 | years | 0.03-0.05 | mg/kg | 0.07-0.9 | mg/kg | 0.11 | mg/kg | 0.17 | mg/kg | 0.24 mg/kg |
| 2-5 | years | 0.11 | mg/kg | 0.17 | mg/kg | 0.24 | mg/kg | 0.30 | mg/kg | — |
| 6-11 | years | 0.11 | mg/kg | 0.17 | mg/kg | 0.24 | mg/kg | 0.30 | mg/kg | — |
| 12 < 18 | years | 0.11 | mg/kg | 0.17 | mg/kg | 0.24 | mg/kg | 0.30 | mg/kg | — |

23. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 22, wherein the administration results in an improvement in the frequency of convulsive/motor seizures.

24. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 23, wherein the administration results in an improvement in one or more of:
frequency of observed countable motor seizures;
number of total seizures;
frequency of non-convulsive seizure;
number of episodes of status epilepticus;
frequency of use of rescue medication; and
number of countable motor seizure-free days.

25. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 24, wherein the administration results in an improvement in the Quality of Life Inventory-Disability (QI-Disability), Subject/Caregiver and Investigator Clinical Global Impression of Improvement (CGI-I), the Investigator Clinical Global Impression of Severity (CGI-S), CaGI-I, Neurobehavioral Evaluation Tool (NET), and/or the 55-item Quality of Life in Childhood Epilepsy Questionnaire (QOLCE-55).

26. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 25, wherein the administration results in at least a 1-point change from baseline in CGI-I and/or CGI-S.

27. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 26, wherein prior to administration, the patient had treatment resistant countable motor seizures with an average of ≥4 observed/countable motor seizures per 4-week period while on stable ASM treatment.

28. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 27, wherein the patient has a DEE but does not have Dravet syndrome or Lennox-Gastaut Syndrome.

29. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 28, wherein prior to administration, the patient had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

30. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 27, wherein the patient has Dravet syndrome.

31. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 30, wherein prior to administration, the patient had:
onset of seizures between 1 and 20 months of age in an otherwise healthy infant; and/or
prolonged generalized tonic-clonic, hemiclonic, myoclonic, tonic, atonic, atypical absence, focal impaired awareness, and/or nonconvulsive status epilepticus.

32. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 30 or 31, wherein prior to administration, the patient had:
an emergence of another seizure type;
prolonged exposure to warm temperatures-induced seizures and/or seizures that were associated with fevers due to illness or vaccines, hot baths, high levels of activity, and/or sudden temperature changes; and/or
seizures induced by strong natural and/or fluorescent lighting.

33. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 30 to 32, wherein prior to administration, the patient had genetic test results consistent with a diagnosis of Dravet syndrome.

34. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 27, wherein the patient has Lennox-Gastaut Syndrome.

35. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to embodiment 34, wherein prior to administration, the patient had:
a history of tonic seizures or tonic/atonic seizures;
more than 1 type of generalized seizure, including but not limited to atypical absence, atonic, myoclonic, focal impaired awareness, generalized tonic-clonic, nonconvulsive status epilepticus, or epileptic spasms;
a history of seizure before 8 years of age;
a history of developmental delay;
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

36. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 35, wherein the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

37. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 36, wherein the Compound 1, or a pharmaceutically acceptable salt thereof, is an HCl salt of Compound 1.

38. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 37, wherein the patient is an infant (less than 2 years of age).

39. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 37, wherein the patient is a child (2 years to less than 12 years).

40. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 37, wherein the patient is an adolescent (12 years to 21 years (up to but not including the 22^{nd} birthday)).

41. The use of (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, for the manufacturing of a medicament according to any one of embodiments 1 to 37, wherein the patient is an adult (22 years or older).

### EXAMPLES

### Example 1: Anti-Seizure Activity in Preclinical Seizure Models

### Rationale

5-HT₂ receptor agonists have demonstrated efficacy for a variety of seizure types and seizure disorders. Compound 1 is a potent, selective 5-HT_{2C} agonist. Proof of concept for the utility of Compound 1 across a range of seizure etiologies was established in zebrafish and mouse model systems. Due to rapid reproductive capacity and ease of genetic manipulation, both zebrafish and mice are highly useful model systems for studying many human diseases. Both have been validated as experimental models for seizures and epilepsy and show sensitivity to many classes of anti-seizure medications.

### Methods

For zebrafish studies, locomotor activity of individual larvae in 96-well plates was tracked with an automated tracking device. Local field potentials were recorded via non-invasive surface recordings from the skin above the optic tectum, and epileptiform activity was quantified.

Experiment 1: Mutations in the human SCN1A gene, which encodes for a voltage-gated sodium channel α-subunit, have been associated with the genetic epilepsy known as Dravet Syndrome. Zebrafish larvae containing mutations in the fish ortholog gene (scnlLab^{-/-}) were treated with Compound 1 or vehicle, and motor behavior and brain epileptiform activity were measured.

Experiment 2: Wild-type zebrafish larvae were treated with ethyl ketopentenoate (EKP), which reduces the synthesis of the inhibitory neurotransmitter GABA, to induce generalized seizures. EKP-treated larvae were exposed to Compound 1, and brain epileptiform activity was recorded.

Experiment 3: Wild-type zebrafish larvae were treated with kainic acid (KA), a cyclic analog of L-glutamate that binds to and activates excitatory glutamate receptors, to induce acute and chronic seizures in zebrafish in a model of temporal lobe epilepsy. KA-treated larvae were exposed to Compound 1, and brain epileptiform activity was recorded.

Experiment 4: Mice were given intravenous pentylenetetrazol (PTZ), an antagonist of the GABA-A receptor, to produce myoclonic and tonic-clonic seizures in a model of generalized epilepsy. Compound 1 was administered orally prior to PTZ administration, and time to the first myoclonic twitch or onset of generalized clonus was recorded.

### Results

Experiment 1: Compound 1 treatment reduced locomotor activity and both the frequency and the mean cumulative duration of epileptiform events (84% and 85%, respectively).

Experiment 2: Compound 1 treatment reduced brain seizure activity an average of 69.1%.

Experiment 3: Compound 1 treatment produced an 82.4% reduction in brain seizure activity.

Experiment 4: Compound 1 administration produced a dose-dependent increase in the time to the first myoclonic twitch and the time of onset to generalized clonus.

The results show that Compound 1 broadly reduces a wide variety of seizure activities stemming from numerous underlying causes including genetic mutations in neuronal sodium channels, reduced GABAergic signaling, and excessive glutamatergic excitation. These data support the usefulness of Compound 1 in treatment of heterogeneous seizure disorders, for example, in DEE patients with heterogeneous underlying pathologies.

### Example 2: Liquid Formulation

A liquid formulation for oral administration was prepared.

| | | | Quantity per |
|---|---|---|---|
| Component | Quality Standard | Function | Unit (g/L) |
| Compound 1 HCl | In house | Active | 3.37^{a} |
| KLEPTOSE ® LINECAPS | USP, EP | Taste masking | 100.00 |
| 17 Pea maltodextrin | | | |
| Glycerin, Synthetic | USP, EP, BP, JP | Thickening agent | 25.00 |
| Sucralose | NF | Sweetener | 10.00 |
| Sodium Benzoate | NF | Preservative | 1.80 |
| Methylparaben | NF | Preservative | 1.30 |
| Cherry Blend FX 770 SD | Vendor CoA | Flavorant | 4.50 |
| FD and C Red No. 40 | Vendor CoA | Colorant | 0.05 |
| Diluted HCl, 10% (w/v) | NF | pH adjustment | 2.28 |
| Purified Water | USP | Solvent, diluent | 897.70 |

EP = European Pharmacopeia (Ph. Eur.); USP = United States Pharmacopeia; BP = British Pharmacopeia; JP = Japanese Pharmacopoeia; CoA = Certificate of Analysis
^{a}Quantity of Compound 1 HCl salt required to provide 3 mg of Compound 1 free base

### Example 3

Predictive Population PK (popPK) Modelling

A model was made to characterize the relationship between the Compound 1 dose, Compound 1 plasma concentrations and Compound 1 CSF concentrations. First, the relationship between Compound 1 dose and plasma levels was characterized. Data from early clinical studies using different formulations were used to identify the absorption characteristics of the formulations as well as the elimination and distribution characteristics of Compound 1. The analysis dataset consisted of a total of 6207 samples obtained from 169 healthy volunteers and 43 patients coming from the clinical studies investigating Compound 1.

The absorption of Compound 1 into plasma was described by first order absorption rate constants with lag times. The relative bioavailability of Compound 1 was found to increase with increasing Compound 1 concentrations in plasma, potentially due to saturation in the pre-systemic metabolism or first pass metabolism in the gut and liver. The distribution and elimination of Compound 1 from plasma was described by a two-compartmental model structure with linear elimination from the central compartment. The predictive population PK (PopPK) model for Compound 1 in plasma was able to describe Phase 1 study data

The popPK model for Compound 1 plasma concentrations was the starting point for including CSF into the final popPK model for Compound 1. Model parameters of the plasma popPK model were fixed and CSF related parameters were estimated based on the available Compound 1 CSF data of Study 102 (see below). A total of 352 samples obtained from 36 subjects in Study 102 was used to build the model.

Compound 1 concentrations in the CSF could be described by the extension of the popPK model using an effect compartment approach. Thereby, a bidirectional exchange of Compound 1 concentrations between the central compartment and the CSF compartment was introduced. Model diagnostics indicated good agreement between observations and simulations in CSF.

Study 102 was an open-label, Phase 1 clinical study evaluating the central nervous system (CNS) pharmacokinetics (PK) and pharmacodynamics (PD) of Compound 1 given orally in healthy volunteers. This study together with data from other studies using Compound 1 in clinical studies was used to model the plasma PK data. The CSF/brain modelling was performed using data from Study 102 only.

The primary objectives of Study 102 were to assess the CNS PK and PD of orally administered Compound 1 in healthy adult male and female participants (n=10 in each Cohort).

Objectives included the characterization of plasma and CSF PK, the characterization of safety and tolerability of doses with titration and taper, and the assessment of the PK-PD relationships between plasma and CSF exposure, and PD endpoints of safety and efficacy, including quantitative electroencephalogram (qEEG) endpoints. Two doses (Cohort 1=6 mg and Cohort 2=12 mg) of Compound 1 given three times daily (TID) were tested over a 16-day period in addition to a screening and follow-up period.

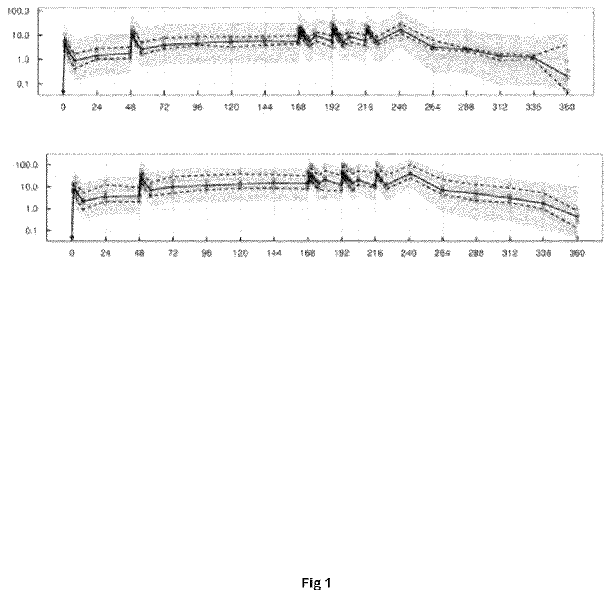

A visual check on the data and the figure illustrate that the final popPK model for Compound 1 reasonably captured the Compound 1 plasma concentrations obtained after oral dosing. The PopPK model for Compound 1 in plasma obtained for patients enrolled in the 102 study accurately described the observed data, indicating that the popPK model is suitable to characterize individual exposures of Compound 1 in plasma. As shown in FIG. 1, the model (as depicted in grey) vs the real data (solid lines) showed that the model encompasses the actual plasma levels of Compound 1.

Exposure-response analyses for efficacy and safety based on data collected from Study 201 (NCT05364021) was performed. Pharmacokinetic data collected from all subjects were included in the analysis. All PK simulated individual exposure metrics (ie, Cavg, Cmax and Ctrough) were highly correlated. Therefore, for efficacy readouts, steady-state exposures (ie, Cavg) were used for the most frequent dose during maintenance phase. The most frequent dose in general was used to simulate the steady-state Cavg data. However, for safety exposure-response analysis, first dose exposures (ie, Cmax) were used since tolerability issues appeared to generally occur in the beginning of the up-titration phase.

Study 201 was a randomized, double-blind, parallel-group, dose-escalation, placebo-controlled study of Compound 1 in adults and adolescents with developmental and epileptic encephalopathies (DEE) with an average of ≥4 observed/countable motor seizures per 4-week period during the 12 weeks before screening while on stable antiseizure medicine.

Subjects were randomized 4:1 to Compound 1 or placebo. The study had a baseline period of 28 days, followed by a 15 day up-titration period during which time subjects will titrate up to their highest tolerated doses, and a 60-day maintenance period. After Day 75, subjects were tapered down over a period of up to 15 days, with a follow-up visit 30 days after last dose.

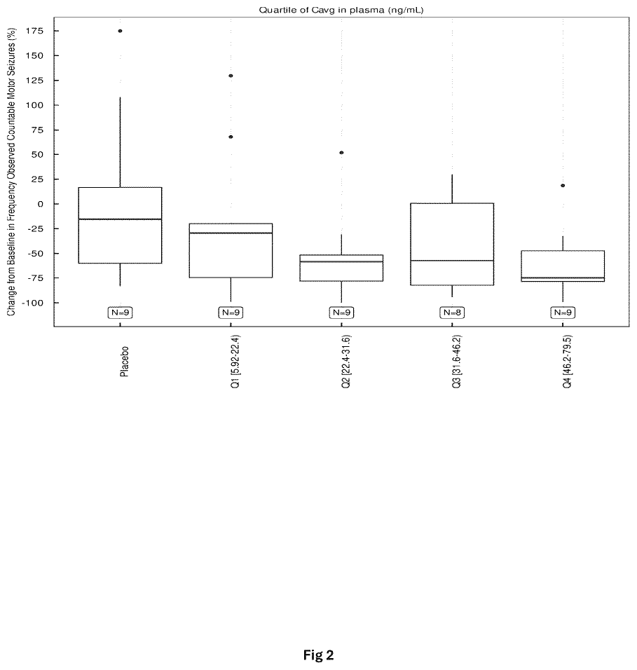

FIG. 2 shows Cavg (average plasma concentration) of Compound 1 in the plasma in steady state against the change in baseline of frequency of observed motor seizure based on data from Study 201. As indicated in FIG. 2, Cavg plasma levels of Compound 1 reaching Q2 (i.e., approximately 22.4 ng/ml and above) were required for attainment of efficacy (efficacy threshold).

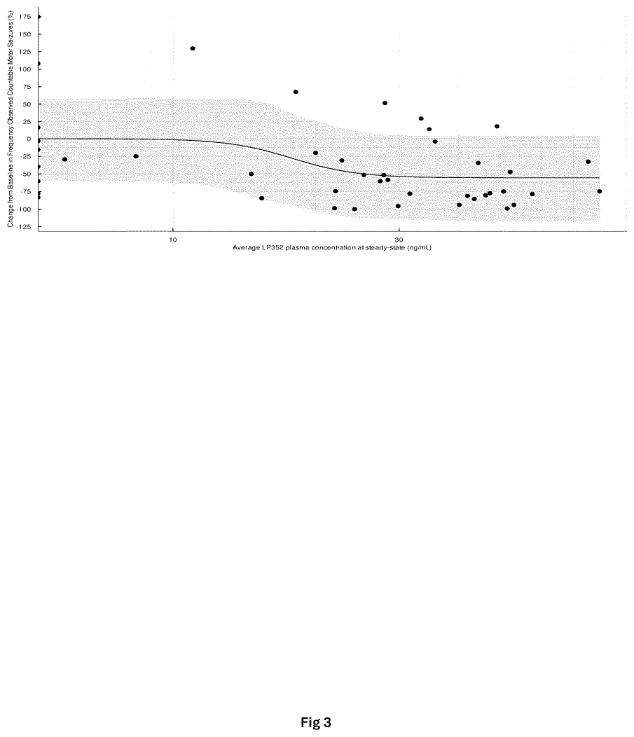

In Table 1 is given the IC50 and the IC70 values for Compound 1 calculated using IMAX model illustrated in FIG. 3.

TABLE 1

| Exposure | IC₅₀ [ng/mL] | IC₇₀ [ng/mL] |
|---|---|---|
| C_{avg} in plasma | 17.9 | 20.4 |

Numbers round to three significant digits. Based on point estimates not considering uncertainty.

IC50 and the IC70 values were calculated on the study data from Study 201 in steady state, and in FIG. 3 is shown the data together with the Imax model prediction of the plasma levels (grey).

As described, Compound 1 has an efficacy threshold when the plasma levels reach 22.4 ng/ml or above as observed using the visualization plots of exposure-response data. However, when using an IMAX model structure for quantitative efficacy threshold values, an IC50 of about 17.9 ng/ml and/or IC70 of about 20.4 ng/ml are required for the observed efficacy of % change in observable countable motor seizures.

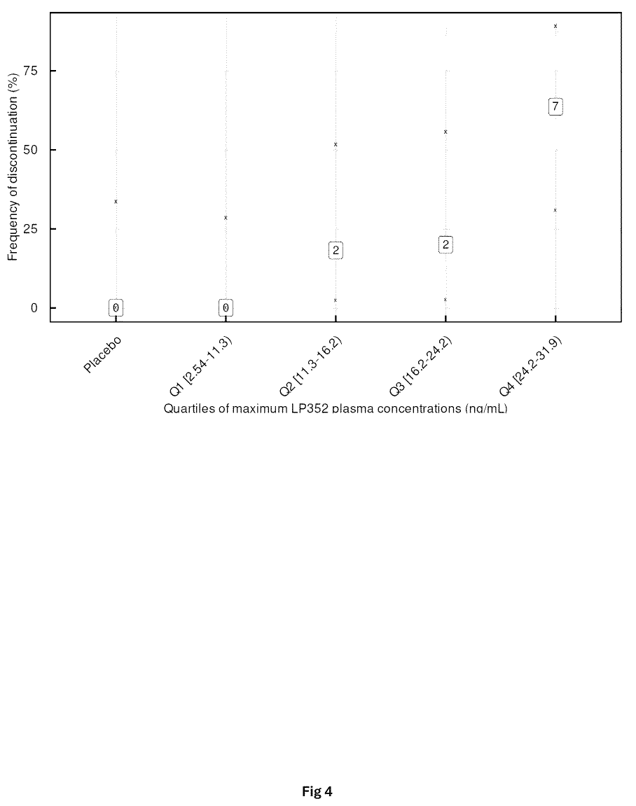

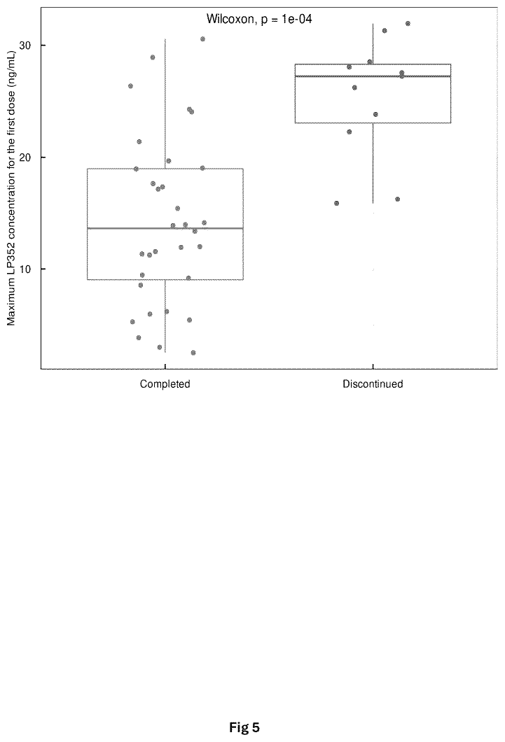

In Study 201 there were 11 subjects on Compound 1 that discontinued the study, but no subjects in the placebo arm that discontinued. The frequency of study discontinuation was found to increase with increasing Compound 1 first dose Cmax. More than 60% of patients in the 4th quartile of Cmax (FIG. 4) discontinued the study, compared to 20% or less in the remaining Cmax quartiles. Furthermore, simulated first dose Cmax for all the study subjects in Study 201 suggested that discontinuation was related to increased Cmax observed in the subjects (FIG. 5).

Thus, dropouts of the study become prominent when the plasma levels reach about 24.2 ng/ml as illustrated in FIG. 4. For treatment with the maximum administered regimen in Study 201, the predicted fraction of adult patients exceeding the IC50 and IC70 obtained from exposure-response modelling were 72.2% and 65.1% (Table 2). It was decided to aim for 65% of patients to exceed the IC50 and 50% of patients to exceed the IC70 for the planned Phase 3 studies.

By comparing the simulated average Compound 1 plasma concentrations at steady-state and target exposures it is evident that about 12 mg TID has an above 50% of patients with an IC50 or IC70. This has been illustrated in Table 2. At starting of dose 6 mg TID gives an opportunity for about 15% of the patients to show efficacy, which keeps increasing at the up-titration step of 9 mg TID and maximizes at the 12 mg TID dose.

TABLE 2
Comparison between Simulated Average Compound 1 Concentrations
for Treatment at Steady-State and Selected Target Exposures

| | % of C_{avg} above | % of C_{avg} above |
|---|---|---|
| Treatment | IC50 FREQMS | IC70 FREQMS |
| 6 mg TID | 16.6 | 12.3 |
| 9 mg TID | 46.8 | 38.8 |
| 12 mg TID | 72.2 | 65.1 |
| 15 mg TID | 87.3 | 82.6 |

Values round to three significant digits. IC50 FREQMS = 17.9 ng/mL, IC70 FREQMS = 20.4 ng/L.
Source: Report FR-006

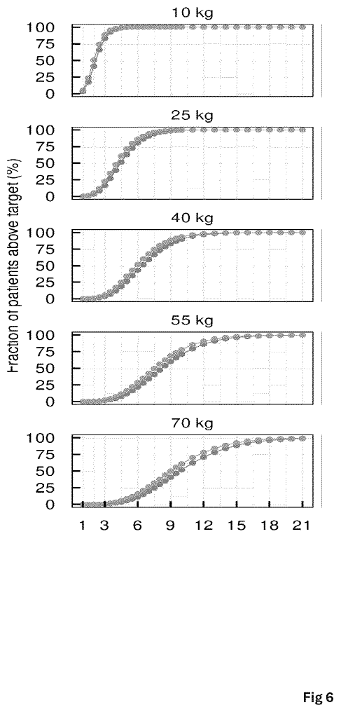

Thus although 12 mg TID appeared to be a good overall dosage, this dosage had many subjects above the target exposure as illustrated in FIG. 6. The fraction of patients above the target (%) vs the TID dosage is given in FIG. 6 for the IC50 and IC70. It is apparent from this illustration that a dosing schedule based on 12 mg TID could be feasible for subjects above about 40 kg, but below 40 kg the fraction of patients above target could be too high.

The lowest maintenance dose for different body weights that achieved the target exposure in at least the target fraction of subjects was identified in FIG. 6.

To support the maintenance phase dosing, combinations of a fixed dose of 12 mg given TID for patients above 40 kg of body weight and body weight-based dose of 0.24 mg/kg were selected for further simulation-based analyses. Both treatments were simulated in subpopulations of varying body weights to determine Compound 1 exposures in plasma.

During simulations, the first dose for body weight-based doses was always 0.11 mg/kg and the first dose of a flat dosing approach was always 6 mg to account for the up-titration scheme of planned studies. Simulated exposures during the maintenance dose were compared to the defined target exposures for efficacy (ie, IC50 and IC70) and a safety threshold that was defined based on the graphical exploration of the exposure-response relation in Study 201 between study discontinuation and first dose Cmax.

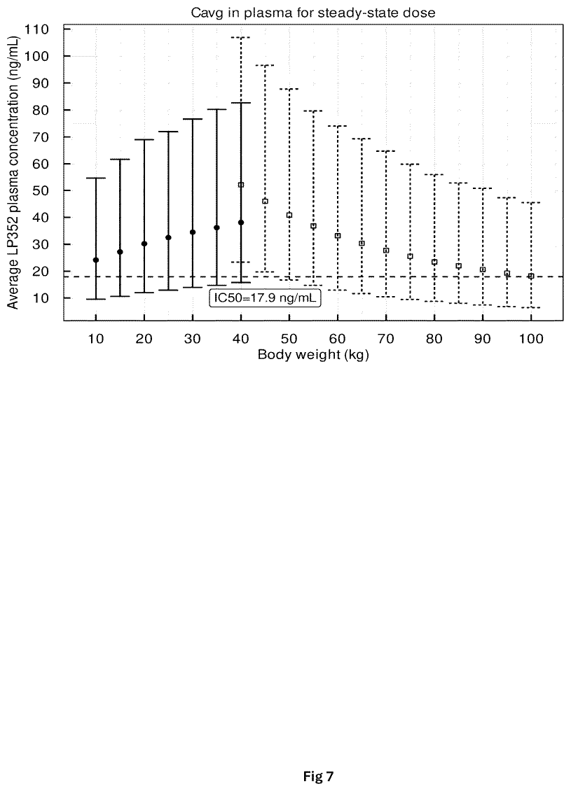

When simulating the dosages at different at different body weights it became apparent that 0.24 mg/kg for subjects below 40 kg was sufficient to maintain an above IC50 plasma concentration of 17.9 ng/ml, and the same was true for 12 mg TID for subjects above 40 kg. This is illustrated in FIG. 7.

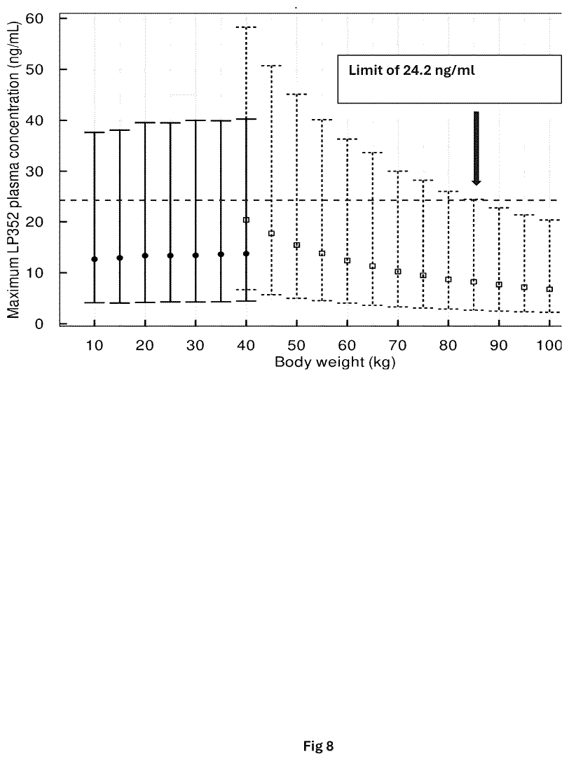

FIG. 8 shows a comparison of the simulated dose of 0.11 mg/kg TID for subjects below 40 kg or 6 mg TID for subjects above 40 kg at different body weights. In the figure is also depicted the 24.2 ng/ml bar to illustrate where the drop out occurs together with the actual patient data (Study 201) and their mean value and standard deviation. It appears the 11 mg/kg TID and 6 mg TID gives a plasma level below the IC50 and IC70, and thus may be insufficient to receive efficacy for some patients. However, for patients either <40 kg or >40 kg, the starting dosages provide low level of drop out/side effects which is directly related for first dose Cmax.

### Example 4

This is a global Phase 3 double-blind, randomized, Placebo-controlled, multicenter study to investigate the efficacy, safety, and tolerability of Compound 1 HCl salt (“Study Drug”) in the treatment of seizures in children and adults with DEE. Randomization will be stratified by 4 categories of age (2 to 5 years, 6 to 11 years, 12 to 17 years, and 18 to 65 years) and 2 categories of country region (Asian, not Asian).

The study consists of the following periods for all participants: Screening (approximately 5 weeks), Titration (3 weeks or Days 1 to 21) and Maintenance (12 weeks or Days 22 to 105). Participants not entering the open-label extension (OLE) will complete Taper (up to 2 weeks) and Follow-Up (4 weeks after completing Taper).

The design of the clinical trial was optimized based on the clinical data from a recently completed Phase 1b/2a study of Study Drug in participants with DEE. The results of the study demonstrated the following:
Study Drug achieved a median seizure reduction of 59.8% in countable motor seizures compared to 17.4% in the Placebo group across the DEE study population.
Study Drug was found to be generally well-tolerated with a favorable safety profile in the 6 to 12 mg three times daily (TID) dose range evaluated.
Study Drug showed low risk of drug-drug interaction potential with several co-administered antiseizure medications (ASMs), supporting dosing of Study Drug without regard to the type or dose of co-administered ASMs.

### Objectives and Endpoints

| OBJECTIVES | ENDPOINTS |
|---|---|
| Primary Objective: | Primary Endpoints: |
| To evaluate the efficacy of Study | Frequency percent change in countable motor seizures |
| Drug in DEE as assessed by | during Treatment (28-day average) compared to Baseline |
| countable motor seizures | (28-day average) as assessed by seizure eDiary |
| Key Secondary Objectives: | Key Secondary Endpoints: |
| To evaluate the safety and | Incidence and severity of TEAEs, including SAEs and |
| tolerability of Study Drug in DEE | AEs leading to discontinuation |
| | Safety laboratory parameters |
| | Physical examination findings |
| | Vital signs |
| | Growth parameters (height and weight) |
| | 12-lead ECGs |
| | C-SSRS responses |
| | PHQ-9 total score and Question 9 score |
| To evaluate the efficacy of Study | 50% responder rate (the percentage of participants with |
| Drug in DEE | a ≥50% reduction in countable motor seizures) during |
| | Treatment (28-day average) compared to Baseline (28-day |
| | average) as assessed by seizure eDiary |
| | Frequency percent change in countable motor seizures |
| | during Maintenance (28-day average) compared to |
| | Baseline (28-day average) as assessed by seizure eDiary |
| To evaluate the efficacy of Study | Frequency percent change in countable total seizures |
| Drug in DEE as assessed by total | during Treatment (28-day average) compared to Baseline |
| seizures | (28-day average) as assessed by seizure eDiary |
| | Frequency percent change in countable total seizures |
| | during Maintenance (28-day average) compared to |
| | Baseline (28-day average) as assessed by seizure eDiary |
| | 50% responder rate for countable total seizures (the |
| | percentage of participants with a ≥50% reduction in |
| | countable total seizures) during Treatment (28-day |
| | average) compared to Baseline (28-day average) as |
| | assessed by seizure eDiary |
| | Frequency percent change in countable motor seizures |
| | during the last 28 days of Maintenance compared to |
| | Baseline (28-day average) as assessed by seizure eDiary |
| | Frequency percent change in countable total seizures |
| | during the last 28 days of Maintenance compared to |
| | Baseline (28-day average) as assessed by seizure eDiary |
| | Frequency change in generalized tonic-clonic seizures |
| | during Treatment (28-day average) compared to Baseline |
| | (28-day average) as assessed by seizure eDiary |
| | Frequency change in generalized tonic-clonic seizures |
| | during Maintenance (28-day average) compared to |
| | Baseline (28-day average) as assessed by seizure eDiary |
| | Within-participant change in number of rescue |
| | medication administrations during Maintenance (28-day |
| | average) compared to Baseline (28-day average) |
| To evaluate the effect of Study | Percentage of participants who achieve 0 to 1 countable |
| Drug on seizure freedom rate in | motor seizures during Maintenance as assessed by seizure |
| DEE | eDiary |
| | Percentage of participants who achieve 0 to 1 countable |
| | total seizures during Maintenance as assessed by seizure |
| | eDiary |
| | 90% responder rate for countable motor seizures (the |
| | percentage of participants with a ≥90% reduction in |
| | countable motor seizures) during Treatment (28-day |
| | average) compared to Baseline (28-day average) as |
| | assessed by seizure eDiary |
| | 90% responder rate for countable total seizures |
| | (percentage of participants with a ≥90% reduction in |
| | countable total seizures) during Treatment (28-day |
| | average) compared to Baseline (28-day average) as |
| | assessed by seizure eDiary |
| | Change in number of days of longest seizure free |
| | interval during Treatment compared to Baseline as |
| | assessed by seizure eDiary |
| | Change in the number of diary days without countable |
| | motor seizures during Maintenance (28-day average) |
| | compared to Baseline (28-day average) as assessed by |
| | seizure eDiary |
| | Change in the number of diary days with complete |
| | seizure freedom during Maintenance (28-day average) |
| | compared to Baseline (28-day average) as assessed by |
| | seizure eDiary |
| To evaluate the effect of Study | Change from Baseline in QI-Disability score at Visit 8 |
| Drug on non-seizure outcomes in | Percent of participants who improved ≥1 point from |
| DEE | Baseline to Visit 8 on the Clinical Global Impression of |
| | Severity (CGI-S) |
| | Change from Baseline in CGI-S |
| | Clinical Global Impression of Improvement(CGI-I) at |
| | Visit 8 |
| | Caregiver Global Impression ofImprovement (CaGI-I) at |
| | Visit 8 |
| To characterize plasma exposures | Pharmacokinetic (PK) simulation of key plasma |
| of Study Drug using PopPK and | exposure metrics (eg, Cmax, Cavg, Ctrough, AUCtau) |
| perform E-R analysis | Conduct plasma E-R analysis with primary efficacy |
| | endpoint and other secondary endpoints |

### Diagnosis and Main Criteria for Inclusion and Exclusion:

The participant population comprises individuals ≥2 to ≤65 years of age with DEE on background ASM and with continued seizure activity.

Specific key inclusion criteria for participants with a diagnosis of DEE include:
Participants who are characterized as having Lennox-Gastaut Syndrome (LGS) must fulfill all of the following criteria:
   Onset of seizures at ≤8 years old
   History of tonic/tonic-atonic seizures plus at least 1 of the following seizure type(s): atypical absence, atonic, myoclonic, focal impaired awareness, generalized tonic-clonic, nonconvulsive status epilepticus, or epileptic spasms
   Presence of developmental plateauing or regression
   History of electroencephalogram (EEG) showing generalized slow (<2.5 Hz) spike-and-wave complexes
Participants who are characterized as having DEE (Other) must fulfill all of the following criteria:
   Does not meet criteria for LGS
   Onset of seizures at ≤5 years old
   Presence of developmental plateauing or regression
   History of multiple seizure types
   History of interictal EEG background showing diffuse or multifocal slowing (with or without epileptiform activity)

Additional specific main inclusion criteria include the following:
The participant has a current occurrence of at least 1 of the following countable motor seizure types: generalized tonic-clonic, tonic, clonic, atonic with truncal involvement, focal motor, and focal to bilateral tonic-clonic.
The participant has demonstrated an average of at least 4 countable motor seizures per month for each of the 3 months prior to Screening.
The participant has been taking 1 to 4 ASMs at a stable dose for at least 4 weeks prior to Screening.
The participant, parent, or caregiver is willing and able (in the judgment of the investigator) to comply with completion of the diaries throughout the study.
The participant must be willing and able to provide written informed consent; in instances where the participant is unable to provide consent, an appropriate legal representative must provide informed consent and the participant will need to assent (as per local regulations) before participation in the study. If the participant cannot provide assent (ie, due to developmental status), the investigator should document why assent was not obtained.

Specific key exclusion criteria include the following:
The participant has a diagnosis of Dravet Syndrome (DS) or has a mutation of the SCN1A gene consistent with DS.
The participant has been admitted to a medical facility for treatment of status epilepticus requiring mechanical ventilation within 3 months prior to Screening.
The participant has a neurodegenerative disorder as indicated by magnetic resonance imaging or genetic testing.
The participant has an acquired lesion/injury unrelated to the primary etiology that could contribute as a secondary cause of seizures.
The participant is receiving one of the following exclusionary medications: fenfluramine or lorcaserin within 28 days of Screening (or failed either in the past), felbamate if not on stable dose for ≥12 months prior to Screening, topiramate or zonisamide if not on stable dose for ≥6 months, and monoamine oxidase (MAO) inhibitors.
The participant has used of any cannabis product or cannabidiol that is not in oral solution/capsule/tablet form, not obtained from a government-approved dispensary, or contains ≥50% THC.
The participant has unstable, clinically significant neurologic (other than the disease being studied; eg, recurrent strokes), psychiatric, cardiovascular (eg, pulmonary arterial hypertension, cardiac valvulopathy, orthostatic hypotension/tachycardia), pulmonary, endocrine disease or other abnormality which may impact the ability of the participant to participate or potentially confound the study results.

### Titration

During Titration, the starting dose and 2 subsequent up-titration doses are determined by participant weight at Day 1:

### Planned Doses and Regimen by Period and Body Weight

| | |
|---|---|
| Period and Weight | Planned Doses |

| | | |
|---|---|---|
| Titration for | Days 1 to 7 | Study Drug 0.11 mg/kg |
| participants | | or Placebo, TID |
| weighing ≤40 kg | Days 8 to 14 | Study Drug 0.17 mg/kg |
| | | or Placebo, TID |
| | Days 15 to 21 | Study Drug 0.24 mg/kg |
| | | or Placebo, TID |

| | |
|---|---|
| Maintenance for | Study Drug at maximum tolerated |
| participants | dose (0.11 mg/kg, 0.17 mg/kg, |
| weighing ≤40 kg | or 0.24 mg/kg) or Placebo, TID |

The target final maintenance dose of study drug (Study Drug or Placebo) for participants with body weight ≤40 kg is the volume corresponding to 0.24 mg/kg Study Drug TID, and the target final maintenance dose of study drug for participants with body weight >40 kg is the volume corresponding to 12 mg Study Drug TID, if tolerated. From the starting dose, participants are up-titrated stepwise on Day 8 and Day 15 regardless of the actual phone visit date, but only after the investigator has determined it is safe to do so.

After completing Titration, participants will continue at their maximum tolerated dose for 12 weeks of Maintenance. During Maintenance, if a participant cannot tolerate their maximum tolerated dose from Titration, they will be withdrawn from the study.

### Efficacy Assessments:

Seizure frequency will be used as the primary measure of the antiseizure activity of Study Drug versus Placebo. During Screening, Titration, and Maintenance, the participant or caregiver will record all seizure occurrences and corresponding seizure type in their seizure eDiary for each day.

Secondary and exploratory assessments include QI-Disability, Clinical Global Impression of Severity (CGI-S), Clinical Global Impression of Improvement (CGI-I), Caregiver Global Impression of Improvement (CaGI-I), Neurobehavior Evaluation Tool (NET), EEG, and caregiver sleep assessment.

Pharmacokinetic and/or Pharmacodynamic Assessments:

Blood samples will be collected for measurement of plasma concentrations of Study Drug and metabolites, at time points specified in the schedule of activities (SoA) and according to procedures specified in the laboratory manual. Plasma samples will be analyzed for Study Drug and metabolite concentrations utilizing a validated liquid chromatography (separation method) with tandem mass spectrometry analyses (LC-MS/MS).

### Safety Assessments:

Each participant will be observed for the entire study duration for safety assessment. Assessments will include adverse events, Hunter Serotonin Toxicity Criteria Review, clinical safety laboratory assessments (including hematology and coagulation, serum chemistry, routine urinalysis, and other Screening tests), physical examinations, vital sign measurements, electrocardiograms (ECGs), and suicidal ideation and behavioral risk monitoring (including Columbia-Suicide Severity Rating Scale and Patient Health Questionnaire-9).

### Example 5

This is a global Phase 3 double-blind, randomized, Placebo-controlled, multicenter study to investigate the efficacy, safety, and tolerability of Study Drug in the treatment of seizures in children and adults with Dravet Syndrome. Randomization will be stratified by 4 categories of age (2 to 5 years, 6 to 11 years, 12 to 17 years, and 18 to 65 years) and 2 categories of country region categories (Asian, not Asian).

The study consists of the following periods for all participants: Screening (approximately 5 weeks), Titration (3 weeks or Days 1 to 21), and Maintenance (12 weeks or Days 22 to 105).

| OBJECTIVES | ENDPOINTS |
|---|---|
| Primary Objective: | Primary Endpoints: |
| To evaluate the efficacy of Study | Frequency percent change in countable motor seizures |
| Drug in DS as assessed by | during Treatment (28-day average) compared to Baseline |
| countable motor seizures | (28-day average) as assessed by seizure eDiary |
| Key Secondary Objectives: | Key Secondary Endpoints: |
| To evaluate the safety and | Incidence and severity of TEAEs, including SAEs and |
| tolerability of Study Drug in DS | AEs leading to discontinuation |
| | Safety laboratory parameters |
| | Physical examination findings |
| | Vital signs |
| | Growth parameters (height and weight) |
| | 12-lead ECGs |
| | C-SSRS responses |
| | PHQ-9 total score and Question 9 score |
| To evaluate the efficacy of Study | 50% responder rate (the percentage of participants with |
| Drug in DS | a ≥50% reduction in countable motor seizures) during |
| | Treatment (28-day average) compared to Baseline (28-day |
| | average) as assessed by seizure eDiary |
| | Frequency percent change in countable motor seizures |
| | during Maintenance (28-day average) compared to |
| | Baseline (28-day average) as assessed by seizure eDiary |
| To evaluate the efficacy of Study | Frequency percent change in countable total seizures |
| Drug in DS as assessed by total | during Treatment (28-day average) compared to Baseline |
| seizures | (28-day average) as assessed by seizure eDiary |
| | Frequency percent change in countable total seizures |
| | during Maintenance (28-day average) compared to |
| | Baseline (28-day average) as assessed by seizure eDiary |
| | 50% responder rate for countable total seizures (the |
| | percentage of participants with a ≥50% reduction in |
| | countable total seizures) during Treatment (28-day |
| | average) compared to Baseline (28-day average) as |
| | assessed by seizure eDiary |
| | Frequency percent change in countable motor seizures |
| | during the last 28 days of Maintenance compared to |
| | Baseline (28-day average) as assessed by seizure eDiary |
| | Frequency percent change in countable total seizures |
| | during the last 28 days of Maintenance compared to |
| | Baseline (28-day average) as assessed by seizure eDiary |
| | Frequency change in generalized tonic-clonic seizures |
| | during Treatment (28-day average) compared to Baseline |
| | (28-day average) as assessed by seizure eDiary |
| | Frequency change in generalized tonic-clonic seizures |
| | during Maintenance (28-day average) compared to |
| | Baseline (28-day average) as assessed by seizure eDiary |
| | Within-participant change in number of rescue |
| | medication administrations during Maintenance (28-day |
| | average) compared to Baseline (28-day average) |
| To evaluate the effect of Study | Percentage of participants who achieve 0 to 1 countable |
| Drug on seizure freedom rate in | motor seizures during Maintenance as assessed by seizure |
| DS | eDiary |
| | Percentage of participants who achieve 0 to 1 countable |
| | total seizures during Maintenance as assessed by seizure |
| | eDiary |
| | 90% responder rate for countable motor seizures (the |
| | percentage of participants with a ≥90% reduction in |
| | countable motor seizures) during Treatment (28-day |
| | average) compared to Baseline (28-day average) as |
| | assessed by seizure eDiary |
| | 90% responder rate for countable total seizures |
| | (percentage of participants with a ≥90% reduction in |
| | countable total seizures) during Treatment (28-day |
| | average) compared to Baseline (28-day average) as |
| | assessed by seizure eDiary |
| | Change in number of days of longest seizure free |
| | interval during Treatment compared to Baseline as |
| | assessed by seizure eDiary |
| | Change in the number of diary days without countable |
| | motor seizures during Maintenance (28-day average) |
| | compared to Baseline (28-day average) as assessed by |
| | seizure eDiary |
| | Change in the number of diary days with complete |
| | seizure freedom during Maintenance (28-day average) |
| | compared to Baseline (28-day average) as assessed by |
| | seizure eDiary |
| To evaluate the effect of Study | Change from Baseline in QI-Disability score at Visit 8 |
| Drug on non-seizure outcomes in | Percent of participants who improved ≥1 point from |
| DS | Baseline to Visit 8 on the Clinical Global Impression of |
| | Severity (CGI-S) |
| | Change from Baseline in CGI-S |
| | Clinical Global Impression of Improvement(CGI-I) at |
| | Visit 8 |
| | Caregiver Global Impression of Improvement (CaGI-I) at |
| | Visit 8 |
| To characterize plasma exposures | Pharmacokinetic (PK) simulation of key plasma |
| of Study Drug using PopPK and | exposure metrics (eg, Cmax, Cavg, Ctrough, AUCtau) |
| perform E-R analysis | Conduct plasma E-R analysis with primary efficacy |
| | endpoint and other secondary endpoints |

### Diagnosis and Main Criteria for Inclusion and Exclusion:

The participant population comprises individuals ≥2 to ≤65 years of age with DS on background ASM and with continued seizure activity.

Specific key inclusion criteria for participants include:
Participants with seizure onset age >1 and <20 months
The participant has a history of at least 1 of the following seizure type(s): prolonged generalized tonic-clonic, hemiclonic, myoclonic, tonic, atonic, atypical absence, focal impaired awareness, and nonconvulsive status epilepticus.
The participant has a current occurrence of at least 1 of the following countable motor seizure types: generalized tonic-clonic, tonic, clonic (hemiclonic or bilateral), atonic with truncal involvement, focal motor, and focal to bilateral tonic-clonic.
The participant has demonstrated an average of at least 4 countable motor seizures per month for the 3 months prior to Screening.
The participant has been taking 1 to 4 ASMs at a stable dose for at least 4 weeks prior to Screening.

Specific key exclusion criteria include the following:
The participant has a history of infantile/epileptic spasms.
The participant has been admitted to a medical facility for treatment of status epilepticus requiring mechanical ventilation within 3 months prior to Screening.
The participant has a neurodegenerative disorder as indicated by magnetic resonance imaging or genetic testing.
The participant has an acquired lesion/injury unrelated to the primary etiology that could contribute as a secondary cause of seizures.
The participant is receiving one of the following exclusionary medications: fenfluramine or lorcaserin within 28 days of Screening (or failed either in the past), felbamate if not on stable dose for ≥12 months prior to Screening, topiramate or zonisamide if not on stable dose for ≥6 months, and monoamine oxidase (MAO) inhibitors.
The participant has used of any cannabis product or cannabidiol that is not in oral solution/capsule/tablet form, not obtained from a government-approved dispensary, or contains ≥50% THC.
The participant has unstable, clinically significant neurologic (other than the disease being studied; e.g., recurrent strokes), psychiatric, cardiovascular (eg, pulmonary arterial hypertension, cardiac valvulopathy, orthostatic hypotension/tachycardia), pulmonary, endocrine disease or other abnormality which may impact the ability of the participant to participate or potentially confound the study results.

### Titration

During Titration, the starting dose and 2 subsequent up-titration doses are determined by participant weight at Day 1:

### Planned Doses and Regimen by Period and Body Weight

| Period and Weight | Planned Doses | |
|---|---|---|
| Titration for | Days 1 to 7 | Study Drug 0.11 mg/kg |
| participants | | or Placebo, TID |
| weighing ≤40 kg | Days 8 to 14 | Study Drug 0.17 mg/kg |
| | | or Placebo, TID |
| | Days 15 to 21 | Study Drug 0.24 mg/kg |
| | | or Placebo, TID |

| | |
|---|---|
| Maintenance for | Study Drug at maximum tolerated |
| participants | dose (0.11 mg/kg, 0.17 mg/kg, |
| weighing ≤40 kg | or 0.24 mg/kg) or Placebo, TID |

The target final maintenance dose of study drug (Study Drug or Placebo) for participants with body weight ≤40 kg is the volume corresponding to 0.24 mg/kg Study Drug TID, and the target final maintenance dose of study drug for participants with body weight >40 kg is the volume corresponding to 12 mg Study Drug TID, if tolerated. From the starting dose, participants are up-titrated stepwise on Day 8 and Day 15 regardless of the actual phone visit date, but only after the investigator has determined it is safe to do so.

After completing Titration, participants will continue at their maximum tolerated dose for 12 weeks of Maintenance. During Maintenance, if a participant cannot tolerate their maximum tolerated dose from Titration, they will be withdrawn from the study.

### Efficacy Assessments:

Seizure frequency will be used as the primary measure of the antiseizure activity of Study Drug versus Placebo. During Screening, Titration, and Maintenance, the participant or caregiver will record all seizure occurrences and corresponding seizure type in their seizure eDiary for each day.

Secondary and exploratory assessments include QI-Disability, Clinical Global Impression of Severity (CGI-S), Clinical Global Impression of Improvement (CGI-I), Caregiver Global Impression of Improvement (CaGI-I), Neurobehavior Evaluation Tool (NET), EEG, and caregiver sleep assessment.

Pharmacokinetic and/or Pharmacodynamic Assessments:

Blood samples will be collected for measurement of plasma concentrations of Study Drug and metabolites, at time points specified in the schedule of activities (SoA) and according to procedures specified in the laboratory manual. Plasma samples will be analyzed for Study Drug and metabolite concentrations utilizing a validated liquid chromatography (separation method) with tandem mass spectrometry analyses (LC-MS/MS).

### Safety Assessments:

Each participant will be observed for the entire study duration for safety assessment. Assessments will include adverse events, Hunter Serotonin Toxicity Criteria Review, clinical safety laboratory assessments (including hematology and coagulation, serum chemistry, routine urinalysis, and other Screening tests), physical examinations, vital sign measurements, electrocardiograms (ECGs), and suicidal ideation and behavioral risk monitoring (including Columbia-Suicide Severity Rating Scale and Patient Health Questionnaire-9).

Although the disclosure has been described with reference to the above examples, it will be understood that modifications and variations are encompassed within the spirit and scope of the disclosure. The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1-4. (canceled)

5. A method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the individual has a body weight of ≤40 kg.

6. The method of claim 5, wherein the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

7. A method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered at a dosage equivalent to from about 0.03 to about 0.3 mg/kg Compound 1 free base three times daily (TID), provided that the individual has a body weight of ≤40 kg.

8. The method of claim 7, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

9. The method of claim 7, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

10. A method of treating or preventing a refractory epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered at a dosage equivalent to from about 0.03 to about 0.3 mg/kg Compound 1 free base three times daily (TID), provided that the individual has a body weight of ≤40 kg.

11. The method of claim 5, wherein Compound 1, or a pharmaceutically acceptable salt thereof, is administered via a titration scheme that comprises the up-titration of Compound 1, or a pharmaceutically acceptable salt thereof, until an optimized dosage is administered.

12. The method of claim 11, wherein the titration scheme comprises administering Compound 1, or a pharmaceutically acceptable salt thereof, at an initial dosage equivalent to about 0.11 mg/kg of Compound 1 TID and, provided that the individual tolerates the initial dosage and that the individual has not had an adequate response, increasing the dosage.

13. The method of claim 12, wherein the individual is less than or equal to 2 years of age.

14. The method of claim 12, wherein the increased dosage is equivalent to about 0.17 mg/kg of Compound 1 TID.

15. The method of claim 12, wherein if the individual does not tolerate the increased dosage, the optimized dosage is the initial dosage.

16. The method of claim 12, wherein if the individual tolerates the increased dosage and if the individual has had an adequate response, the optimized dosage is the increased dosage.

17. The method of claim 11, wherein the titration scheme comprises further increasing the dosage, provided that the individual tolerates the increased dosage and that the individual has not had an adequate response.

18. The method of claim 16, wherein the further increased dosage is equivalent to about 0.24 mg/kg Compound 1 free base three times daily (TID).

19. The method of claim 17, wherein if the individual tolerates the further increased dosage and if the individual has had an adequate response, the optimized dosage is the further increased dosage or maintenance dose selected from 0.11 mg/kg, 0.17 mg/kg or 0.24 mg/kg.

20. The method of claim 11, further comprising administering the optimized dosage of Compound 1, or a pharmaceutically acceptable salt thereof, to the individual.

21. The method of claim 11, wherein the titration scheme further comprises the down-titration of Compound 1, or a pharmaceutically acceptable salt thereof.

22. The method of claim 11, wherein the patient is ≤40 kg and Compound 1, or a pharmaceutically acceptable salt thereof, is administered TID at a dosage equivalent to Compound 1 free base according to the following schedule:

| Age | | Initial dose | | Up- | | Up- | | Up- | | Up- |
|---|---|---|---|---|---|---|---|---|---|---|
| stratification* | | (TID) | | titration | | titration | | titration | | titration |
| 0-<2 | years | 0.03-0.05 | mg/kg | 0.07-0.9 | mg/kg | 0.11 | mg/kg | 0.17 | mg/kg | 0.24 mg/kg |
| 2-5 | years | 0.11 | mg/kg | 0.17 | mg/kg | 0.24 | mg/kg | 0.30 | mg/kg | — |
| 6-11 | years | 0.11 | mg/kg | 0.17 | mg/kg | 0.24 | mg/kg | 0.30 | mg/kg | — |
| 12 < 18 | years | 0.11 | mg/kg | 0.17 | mg/kg | 0.24 | mg/kg | 0.30 | mg/kg | — |
